(19) United States
(12) Patent Application Publication
(10) Pub. No.: US 2026/0146086 A1
(43) Pub. Date: May 28, 2026

(54) HETEROGENEITY OF HUMAN CD79 AND SYNERGISTIC ENHANCEMENT OF ANTITUMOR ACTIVITY BY CO-TARGETING OF CD79B AND CD79A IN THE THERAPY OF B CELL TUMORS

(71) Applicant: Health Research, Inc., Buffalo, NY (US)

(72) Inventor: Ben SEON, Williamsville, NY (US)

(21) Appl. No.: 19/120,206

(22) PCT Filed: Oct. 12, 2023
(86) PCT No.: PCT/US2023/076721

§ 371 Date: Apr. 10, 2025

Related U.S. Application Data
(60) Provisional application No. 63/415,434, filed on Oct. 12, 2022.

(51) Int. Cl. C07K 16/28; A61K 39/00; A61P 35/02

(52) U.S. Cl. CPC C07K 16/2803; A61P 35/02; A61K 2039/505

(57) ABSTRACT

Provided are compositions and methods that relate to therapy of B cell diseases and include discovery of novel molecular heterogeneity of the signaling component (i.e., CD79) of human B cell antigen receptor (BCR). The method includes co-administering antibodies or antigen binding fragments thereof that specifically bind CD79a and CD79b to provide synergistic therapy of B cell tumors. The method may further include administering an antibody that specifically binds to an immune checkpoint molecule, such as programmed cell death protein 1 (PD-1).

### CROSS REFERENCE TO RELATED APPLICATIONS

This application claims priority to U.S. provisional patent application No. 63/415,434, filed Oct. 12, 2022, the entire disclosure of which is incorporated herein by reference.

### FIELD

The present disclosure is related to the discovery of novel molecular heterogeneity of human CD79, the signaling component of the human B cell antigen receptor, and enhancement of CD79-targeted therapy of human B cell tumors.

### RELATED INFORMATION

### 1. CD79

f (mlg) and the signal-transducing CD79a/CD79b (also termed Igα/Igβ) heterodimer. The antigen-binding mlg and the signal-transducing CD79a/CD79b dimer is associated non-covalently whereas CD79a/CD79b is a disulfide-linked heterodimer (Hombach et al., 1990, Nature 343:760-762; Okazaki et al., 1993, Blood 81:84-94, Vasile et al., 1994, Mol. Immunol. 31:419-427; Sieger et al., 2006, Int. Immunol. 18:1385-1396). CD79a and CD79b have a similar structure in the form of an extracellular Ig domain, a short linker, a conserved transmembrane (TM) domain, and a cytoplasmic tail carrying an immunoreceptor tyrosin-based activation motif (ITAM) that connects the BCR to the protein tyrosine kinase Syk (Hombach et al., 1990, supra; Radaev et al., 2010, Structure 18:934-943; Young et al., 2019, Immunol. Rev. 291:190-213; Gottwick et al., 2019, Proc. Natl. Acad. Sci. 116:13468-13473). Upon antigen ligation, the spleen tyrosine kinase (Syk) phosphorylates and interacts with the ITAM tyrosines of CD79a and CD79b (Rolli et al., 2002, Mol. Cell 10:1057-1069). The resulting ITAM/Syk complex amplifies the BCR signal and connects the BCR to several down-stream signaling pathways leading to activation, proliferation and differentiation of B cells (Johnson et al., 1995, J. Immunol. 155:4596-4603; Werner et al., 2010, Immunol. Rev. 237:55-71; Young et al., 2019, supra).

2. Monoclonal Antibodies (mAbs) to Human CD79

At the 5th International Conference and Workshop on Human Leukocyte Differentiation Antigens (3-7 Nov. 1993, Boston USA), CD79 was assigned to the human homologue of mouse Igα/Igβ [B23 CD79 Workshop report, P Engel, N Wagner, TF Tedder in Leucocyte Typing V: White Cell Differentiation Antigens (Edited by SF Schlossman et al.) Oxford University Press, Oxford, 1995]. mAb SN8 was determined to be the only mAb that reacted with an extracellular epitope of CD79, whereas all of the other three anti-CD79 mAbs were determined to react with intracellular epitopes of CD79 which indicated the limited utility of these three mAbs for the CD79-targeted therapy of B cell diseases. SN8 was determined to react with CD79b (Engel et al., 1993, supra; Vasile et al., 1994, supra). Subsequently mAb SN8 was shown to be useful for diagnosis and classification of various B cell malignancies including non-Hodgkin's lymphoma (NHL), prolymphocytic leukemia (PLL), acute lymphoblastic leukemia (ALL), chronic lymphocytic leukemia (CLL) and hairy cell leukemia (Okazaki et al, 1993, supra; Zomas et al., 1996, Leukemia 10:1966-1970; Astsaturov et al., 1996, Leukemia 10:769-773; Moreau et al., 1997, Am. J. Clin. Pathol. 108:378-382). SN8a was determined to react with an extracellular epitope of CD79b as SN8 while SN8b appeared to react with an extracellular epitope of CD79a (Okazaki et al., 1993, supra; Vasile et al., 1994, supra. Conjugates of mAb SN8, mAb SN8a and mAb SN8b with deglycosylated ricin A-chain were highly effective for targeted killing of CD79-expressing malignant B cells in an in vitro assay which showed that these immunoconjugates were effectively internalized into the malignant B cells after these mAbs bound to the respective extracellular epitopes of CD79 (Okazaki et al., 1993, supra). SN8 was derived from hybridoma clone 3A2-2E7 while SN8b was derived from hybridoma clone Q6-1D5 (Okazaki et al., 1993, supra).

### 3. Immune Checkpoint Blockade (ICB) Strategy to Enhance Immunotherapy of Cancer

Unprecedented advances have been made in cancer treatment by use of ICB although the responses are limited to a subset of patients (Morad et al., 2021, Cell 184:5309-5337). ICB strategies of tumor therapy involve modulation of T cell activation against tumor cells (Topalian et al., 2012, N. Engl. J. Med. 366:2443-2454; Pardoll, D M, 2012, Nat. Rev. Cancer 12:252-264; Ribas, A, 2012, N. Engl. J. Med. 366:2517-2519; Wei et al., 2017, Cell 170:1120-1133; Morad et al., 2021, supra). In the present work, we combined anti-CD79a mAb and/or anti-CD79b mAb with ICB antibody for the therapy of human B cell tumor in SCID mice. We have used anti-programmed cell death protein 1 (PD-1) antibody as the ICB agent. SCID mice are deficient of T cells and B cells but contain natural killer (NK) cells (Bosma et al., 1983, Nature 301:527-530; Welsh et al., 1991, J. Exp. Med. 173:1053-1063). Despite the lack of T cells in the SCID mice, anti-PD-1 antibody strongly potentiated antitumor activity of anti-CD79b mAb and anti-CD79a mAb plus anti-CD79b mAb in SCID mice. Furthermore, the potentiation by anti-PD-1 antibody did not alter the synergistic activation of the therapeutic activity by combination of anti-CD79a mAb and anti-CD79b mAb compared with therapy with anti-CD79a mAb alone and anti-CD79b mAb alone.

4. Animal models for CD79-Targeted Therapy of B Cell Tumors

The use of mouse models of human tumors has been accepted and validated as a model for the evaluation of therapeutic agents because the models have been shown to be predictive of the effectiveness of therapeutic agents. Mouse models of various human tumors have been used for therapeutic studies by a large number of investigators. These human tumors include B-lineage acute lymphoblastic leukemia (ALL) (Hara et al., 1988, Cancer Res. 48:4673-4680; Luo and Seon, 1990, J. Immunol. 145:1974-1982; Yoshida et al., 1997, Cancer Res. 57:678-685), T cell ALL (Hara and Seon, 1987, Proc. Natl. Acad. Sci. 84:3390-3394; Yokota et al., 1990, Cancer Res. 50:32-37), breast cancer (Seon et al., 1997, Clin. Caner Res. 3:1031-1044; Matsuno et al., 1999, Clin. Cancer Res. 5:371-382), and B cell lymphoma (Dornan et al., 2009, Blood 114:2721-2729). The therapeutic studies of a drug conjugate of humanized anti-CD79b mAb SN8 in SCID mice bearing human B lymphoma provided a basis for the drug conjugate, later named Polatuzumab vedotin (PV), for the therapy of adult patients with relapsed or refractory (R/R) diffuse large B cell lymphoma (DLBCL) which was recently approved by FDA. Nevertheless, there is an ongoing and unmet need for compositions and methods for therapy of B Cell tumors. The present invention is related to this need.

### BRIEF SUMMARY

The present disclosure is related to compositions and methods for use in therapy of cancers that express B cell antigen receptors. In particular, the present disclosure is related to the discovery of molecular heterogeneity of human CD79 that is related to the non-homogeneous response to CD79a-targeted therapy and CD79b-targeted therapy. Furthermore, the present disclosure is related to the molecular basis of the discovery that the synergistic potentiation of antitumor activity by dual targeting of CD79a and CD79b in the therapy of B cell tumors can be achieved. The disclosure shows that a combination of anti-programmed cell death protein 1 (PD-1) antibody with anti-CD79b mAb or anti-CD79a mAb plus anti-CD79b mAb strongly enhanced antitumor activity of each of the combination components. Furthermore, the synergistic effect of the combined targeting of CD79a and CD79b is maintained after addition of the anti-PD-1 antibody. The disclosure is further related to development of methods for distinguishing patients to select patients for dual targeting of CD79a and CD79b. In addition, the present disclosure is related to the discovery that CD79a can be effectively targeted for therapy of B cell tumors expressing CD79 as well as CD79b. This includes generation of conjugates of anti-CD79a antibody with other anticancer agents such as drugs which can be effectively used for therapy of B cell tumors.

The anti-CD79b mAb SN8-derived drug conjugate, termed polatuzumab vedotin (PV; or named Polivy), is a major component of a therapeutic regimen that was approved by the US Food and Drug Administration (FDA) for therapy of adult patients with relapsed or refractory diffuse large B-cell lymphoma (DLBCL). DLBCL is the most common form of lymphoma. In addition, a recent double-blind, placebo-controlled international phase III clinical trial to evaluate the anti-CD79b PV-containing therapeutic regimen, termed pola-R-CHP (PV-rituximab-cyclophosphamide, doxorubicin, prednisone), in patients with previously untreated intermediate-risk or high-risk DLBCL revealed that the pola-R-CHP regimen is more effective for lowering disease progression, relapse or death of the patients than the standard of care regimen, namely R-CHOP (Tilly et al., 2021, N. Engl. J. Med. 386:351-363). PV in combination with R-CHP was approved on Apr. 19, 2023 by US FDA for therapy of previously untreated patients with DLBCL or high grade lymphoma. The data presented in this disclosure indicate that antitumor activity of anti-CD79b drug conjugate PV can be further potentiated by combining with anti-CD79a mAb or drug conjugate in the therapy of patients with DLBCL and other B cell lymphoma.

Experimental data of the synergistic effect of the dual targeting of CD79a and CD79b of B cell tumors is presented below.

Results in this disclosure were obtained using two animal models. One of the models is animals bears subcutaneous tumors of DLBCL that were treated with systemic (intravenous) administration of a control, anti-CD79a mAb, anti-CD79b mAb, or anti-CD79a mAb plus anti-CD79b mAb in the absence or presence of anti-PD-1 antibody. The other animal model is mice bearing systemic tumors of mature B cell ALL that were treated with systemic administration of control, anti-CD79a mAb, anti-CD79b mAb or anti-CD79a mAb plus anti-CD79b mAb. Results of these animal models confirm our invention. These results are presented below.

In one aspect the disclosure demonstrates the molecular basis of heterogeneity of CD79, the signaling component of the B cell antigen receptor (BCR) and the molecular basis of synergistic enhancement of CD79-targeted therapy of B cell tumors by dual targeting of CD79a and CD79b.

In one aspect, the disclosure provides a new finding that anti-programmed cell death protein 1 (PD-1) antibody strongly potentiates antitumor activity of anti-CD79b mAb and anti-CD79a mAb plus anti-CD79b mAb in a T cell-independent manner. Antitumor immunity encompasses tumor antigen presentation to T cells via antigen presenting cells (APC) or tumor cells, followed by T cell activation against tumor cells, which involves a number of costimulatory and inhibitory molecules including CD28, CTLA-4 (cytotoxic T lymphocyte-associated protein 4) and PD-1 (Morad et al., 2021, supra). However, in the present disclosure, we found that anti-PD-1 antibody can potentiate antitumor activities of anti-CD79b mAb and anti-CD79a mAb plus anti-CD79b mAb in the absence of T cells. In the anti-PD-1 antibody-mediated potentiation, the synergistic enhancement of dual targeted therapy of CD79a and CD79b is maintained.

In another aspect the disclosure demonstrates that the CD79a-targeted therapy can be effective for therapy of B cell tumors in addition to the CD79b-targeted therapy of B cell tumors. In this disclosure, we show that CD79a-targeted therapy can be effective for the suppression of B cell tumors by use of two different animal models.

The superiority of the dual targeting of CD79a and CD79b to the single targeting of CD79a or CD79b is also demonstrated by use of the two animal models.

One of the animal models is mice bears subcutaneous tumor of human B cell lymphoma. Growth of this tumor is suppressed by systemic intravenous administration of anti-CD79a monoclonal antibody (mAb), termed SN8b, anti-CD79b mAb, termed SN8, or SN8b plus SN8. Tumor growth suppression is followed by measuring tumor size and/or the extension of survival of the tumor-bearing mice compared with controls.

Another animal model is mice bears systemic tumors of mature B cell acute lymphoblastic leukemia (B ALL). Antitumor efficacy of SN8b, SN8 or SN8b plus SN8 in mice bearing this B ALL tumor is measured by following the extension of the survival of the tumor-bearing mice compared with controls after systemic intravenous administration of the mAbs. In both tumor model experiments, anti-CD79a mAb SN8b shows significant antitumor activity. Furthermore, superiority of dual targeting of CD79a and CD79b to the single targeting of either CD79a or CD79b is demonstrated in both animal models.

In another aspect the disclosure provides the described mAbs conjugated to antitumor agents to provide stronger CD79-targeted agents for effective therapy of B cell tumors. The humanized version of anti-CD79b mAb SN8 was conjugated with monomethylauristatin E (MMAE) (Dornan et al., 2009, Blood 114:2721-2729), and named Polatuzumab vedotin (PV) (Sehn et al., 2020, J. Clin. Oncol. 38:155-165). Polatuzumab vedotin (PV) has been successfully tested in several clinical trials (e.g., Sehn et al., 2020, supra). The FDA granted accelerated approval to PV in combination with bendamustine and a rituximab product for adult patients with relapsed or refractory diffused large B-cell lymphoma (DLBCL) after at least two prior therapies.

In addition, more recently PV in combination with R-CHP was approved by US FDA for therapy of previously untreated patients with DLBCL or high grade B cell lymphoma.

Another aspect of the disclosure comprises use of CD79a-directed antibody-drug conjugate and CD79b-directed antibody-drug conjugate for dual CD79a-targeted and CD79b-targeted therapy of B cell tumors expressing CD79.

Another aspect of the disclosure is the use of anti-CD79a mAb SN8b-drug conjugate and anti-CD79b mAb SN8-drug conjugate for dual CD79a-targeted and CD79b-targeted therapy of B cell tumors.

Another aspect of the disclosure is the use of chimeric antigen receptor (CAR) T cells containing antigen-binding fragments of antibody (e.g., single chain Fv) of anti-CD79a mAb and/or anti-CD79b mAb for therapy of patients with B cell tumors.

### BRIEF DESCRIPTION OF FIGURES

FIG. 1 depicts a flow cytometry analysis that demonstrates specific blocking of the binding of SN8b-Alexa Fluoro 647 (SN8b-Ax647) to BALL-1 cells by incubation with serial concentrations of autologous mAb SN8b (a positive control) and anti-CD79a mAb ZL7-4 (from Invitrogen) but not by any of anti-CD79b mAb SN8, anti-CD79b mAb SN8a and an isotype-matched control IgG CCL130 (IgG1-K). These results in addition to the previous results (Okazaki et al., 1993, supra) show that mAb SN8b defines a cell surface epitope of CD79a.

FIGS. 2A-2B depict SDS-PAGE analysis of CD79a/CD79b complex or CD79a and CD79b molecules that are generated by reduction of the disulfide bonds between CD79a and CD79b molecules. Data in Section A are those of SDS-PAGE analysis of the immunoprecipitates from Experiment 1 of Table 1, whereas data in Section B are those of SDS-PAGE analysis of the immunoprecipitates from Experiment 2 of Table 1. In Section A, an 85 kDa CD79a/CD79b complex (non-reduced, upper panel) or a 49 kDa CD79a molecule and a 40 kDa CD79b molecule (reduced, lower panel) are detected after immunoprecipitation with anti-CD79b mAb SN8 (panel 1) and anti-CD79a mAb SN8b (panel 2) from the control IgG (MOPC)-pansorbin pre-cleared ¹²⁵I-BPLL antigen preparation. From the SN8-pansorbin pre-cleared ¹²⁵I-BPLL antigen preparation, the CD79a/CD79b complex is not detected in the immunoprecipitate of either SN8 (lane 4) or MOPC (lane 6), whereas both CD79a/CD79b complex (non-reduced, upper panel) and CD79a and CD79b molecules (reduced, lower panel) are detected in the immunoprecipitate with SN8b (lane 5). From the SN8b-pansorbin pre-cleared ¹²⁵I-BPLL antigen preparation, the CD79a/CD79b complex is not detected in the immunoprecipitate of either SN8b (lane 8) or MOPC (lane 9), whereas both the CD79a/CD79b complex (non-reduced, upper panel) and CD79a and CD79b molecules (reduced, lower panel) are detected in the immunoprecipitate of SN8 (lane 7). These results are consistent with the results of Table 1 and show the existence of molecular heterogeneity of CD79 complex. It should be noted that anti-CD79b mAb SN8 and anti-CD79a mAb SN8b do not interfere each other in their binding with CD79 complex in the competitive binding assays (FIG. 1 in this patent application; Okazaki et al., 1993, Blood 81:84-94). Results of the Section B (from Experiment 2) are consistent with the described results of the Section A (from Experiment 1).

FIG. 3A depicts reactivity of anti-CD79b mAb SN8 and anti-CD79a mAb SN8b with 6 human leukemia/lymphoma cell lines in a two-dimensional flow cytometry analysis. For the present analysis, anti-CD79b mAb SN8 was labeled with phycoerythrin (PE) while anti-CD79a mAb SN8b was labeled with Alexa Fluoro 647 (AxF1647). FITC-labeled anti-CD45 mAb (from eBioscience) is included in the assay as a leucocyte marker. Neither SN8 nor SN8b shows significant reactivity with MOLT-4 T acute lymphoblastic leukemia (ALL) cells and KM-3 immature B-lineage ALL cells. In contrast, both SN8 and SN8b reacted with majority of cells of BALL-1 (mature B ALL), SU-DHL-4 (diffuse large B cell lymphoma, DLBCL), Karpas-422 (K422, DLBCL) and TMD8 (DLBCL). In a simultaneous staining experiment with SN8 and SN8b, the majority of these four cell lines reacted with both SN8 and SN8b. However, these results do not indicate that anti-CD79b mAb SN8 and anti-CD79a mAb SN8b react with CD79b and CD79a of the same CD79 molecules, but the results of Table 1 and FIGS. 2A-B indicate that SN8 and SN8a are reacting with CD79b and CD79a of different CD79 molecules. It should be noted that BALL-1 cells express 5.9×10⁴ SN8 epitopes and 5.7×10⁴ SN8b epitopes per cell on the cell surface (Okazaki et al., 1993, Blood, supra).

FIG. 3B depicts the continued tests of reactivity of PE-labeled anti-CD79b mAb SN8 and AxF1647-labeled anti-CD79a mAb SN8b with 6 additional human leukemia-lymphoma cell lines; in the assay FITC-labeled anti-CD45 mAb is included as a positive control for the leucocytes. These 6 cell lines include BALM-3 (B lymphoma), U698-M (B lymphoma), Daudi (Burkitt's lymphoma), Ramos (Burkitt's lymphoma), BALL-la (in vivo adapted BALL-1) and MO-1043 (B chronic lymphocytic leukemia, B CLL). The percentage of the SN8b-positive cells is lower than that of the SN8-positive cells for BALM-3, U698-M and Ramos. Consequently, the percentage of the CD79b/CD79a double positive cells for these 3 cell lines is 64.47%, 69.04% and 62.82%, respectively, and is lower than that of SN8-positive cells, i.e., 89.09%, 88.73% and 83.23%, respectively. Dual targeting of CD79a and CD79b in these malignant B cells will be less effective compared with the dual targeting of BALL-1, SU-DHL-4, K422 and TMD8 of FIG. 3A, and also compared with the dual targeting of Daudi and BALL-la of FIG. 3B. In contrast, Daudi expresses a similar level of CD79b (91.61%) and CD79a (94.05%) and consequently expresses a similar level (93.36%) of double-positive cells. BALL-la, an in vivo adapted BALL-1 (Kawata et al., 1994, Cancer Res. 54:2688-2694), express similar levels of CD79b and CD79a to BALL-1 (see FIG. 3B). MO1043 is an established B CLL cell line (Kawata et al., 1993, Leukemia Res. 17:883-894) and express low levels of CD79b and CD79a.

FIG. 3C depicts reactivity of PE-labeled anti-CD79b mAb SN8, Alexa Fluoro 647 (AxF1647)-labeled anti-CD79a mAb SN8b and FITC-labeled anti-CD45 mAb (control) with normal human peripheral blood lymphocytes (NH PBL), prolymphocytic leukemia (PLL) lymphocytes, non-Hodgkin's lymphoma (NHL) lymphocytes from 2 patients (#1 and 2) and chronic lymphocytic leukemia (CLL) lymphocytes from 2 patients (#1 and 2). Eighteen % of NH PBL reacts with the PE-SN8 and the AxF1647-SN8b, respectively, and 17% of the cells are double-positive for SN8 and SN8b. The PE-SN8 and the AxF1647-SN8b react with 98.15% and 98.17% of the PLL cells, respectively, and the same number (98.20%) of the cells are double positive for SN8 (anti-CD79b) and SN8b (anti-CD79a). Over 90% of the No. 1 NHL cells reacts with PE-SN8 and AxF1647-SN8b, respectively, and the similar number (92.61%) of the cells are double positive for SN8 and SN8b. In contrast, only 50.42% and 47.62% of the No. 2 NHL cells react with PE-SN8 and AxF1647-SN8b, respectively, and only 23.71% of the NHL cells are double positive for SN8 and SN8b. The results indicate that #1 NHL cells can be targeted better by dual targeting with SN8 and SN8b compared with #2 NHL cells. Anti-CD79a AxF1647-SN8b reacts with higher percentage of CLL cells (#1 and 2) than anti-CD79b PE-SN8 (48.50% vs 21.26% for #1 and 52.54% vs 45.66% for #2) and the number of the double-positive cells are low, i.e., 15.88% for #1 and 31.29% for #2. In general CLL cells are not good candidates for CD79-targeted therapy. Nevertheless, some CLL cells such as #2 CLL cells could be candidates for CD79-targeted therapy by dual targeting of CD79a and CD79b.

FIG. 4 depicts synergistic suppression of growth of K422 human DLBCL tumor in SCID mice by combined use of anti-CD79 mAbs and anti-programmed cell death protein 1 (PD-1) antibody. The antitumor activities of anti-CD79 mAbs and anti-PD-1 antibody are measured by decrease in tumor size after intravenous (i.v.) administration of mAbs and antibody into tumor-bearing SCID mice. Immune checkpoint blockade strategies for tumor therapy including anti-PD-1 antibody therapy are known to involve modulation of cytotoxic T cell activation against tumor cells (Topalian et al., 2012, supura; Pardoll, 2012; supra; Ribas, 2012, supra; Wei et al., 2017, supra; Moral et al, 2021, supra). On the other hand, SCID mice are deficient of T and B cells (Bosma et al., 1983, supra). We (see FIG. 8) and others (Welsh et al., 1991, J. Exp. Med., 173:1053-1063) found that SCID mice possess natural killer (NK) cells which may contribute to PD-1/PD-L1 blockade (Hsu et al., 2018, J. Clin. Invest. 128:4654-4668). In the present and the following studies, we found that anti-PD-1 antibody shows strong antitumor activity. Analysis of synergy by dual targeting of CD79a and CD79b of B cell tumor was performed with the help of the Bioanalytics, Metabolomics and Pharmacokinetics (BMPK) Shared Resource of Roswell Park Comprehensive Cancer Center. The synergy analysis was performed by use of the statistical determination of synergy (Demidenko and Miller, 2019, PLOS ONE 14 (11): e0224137). The analysis demonstrated that anti-CD79a mAb SN8b+anti-CD79b mAb SN8 show strong statistically significant synergistic antitumor activity compared with SN8b alone and SN8 alone in the presence of anti-PD-1 antibody (P=0.00005; Control: CCL130+anti-PD-1 antibody). Furthermore, anti-PD-1 antibody strongly potentiates antitumor activities of SN8b, SN8 and SN8b+SN8, respectively (see below FIGS. 5A-5B).

FIGS. 5A-5B depict strong suppression of K422 human diffuse large B cell lymphoma (DLBCL) in SCID mice by combined use of anti-CD79a mAb SN8b and anti-CD79b mAb SN8 in the absence or in the presence of anti-PD-1 antibody. In FIG. 5A in which mice were followed for 27 days after tumor-inoculation, all mice survived (n=6 for each group, except for n=4 for the isotype-type matched control IgG, IgG1-κ, CCL130). In FIG. 5B, in which the tumor-bearing mice were followed for 39 days, all of the control group mice died but all mice of the 6 other groups survived, and tumor size of the surviving mice are compared. Antitumor activities of SN8b and SN8 are strongly potentiated by combination between them compared with SN8b alone and SN8 alone. This statistically significant potentiation is observed either in the absence or in the presence of anti-PD-1 antibody. On the other hand, addition of anti-PD-1 antibody to SN8b, SN8 or SN8b+SN8 strongly potentiated antitumor activity in each case.

FIGS. 6A-6B depict effect of administration of anti-CD79a mAb SN8b, anti-CD79b mAb SN8 or SN8b+SN8 on the survival of SCID mice bearing K422 human DLBCL. FIG. 6A depicts extension of survival of K422 tumor bearing mice by intravenous (i.v.) administration of SN8b, SN8 or SN8b+SN8 in the absence or in the presence of anti-PD-1 antibody, an immune checkpoint blocker. CCL130 is an isotype-matched control IgG (IgG1-κ). Combined administration of SN8b and SN8 strongly enhanced the extension of survival by SN8b alone or SN8 alone in the absence of anti-PD-1 antibody (P<0.01, ** and P<0.01, **, respectively). Furthermore, SN8b and SN8 acted synergistically in the extension of the survival as demonstrated by statistical determination of synergy (Demidenko and Miller, 2019, supra) (P=0.03; Control: CCL130). Addition of anti-PD-1 antibody to SN8 or SN8+SN8b enhanced the extension of survival (* P<0.05 and ** P<0.01, respectively). The effect of anti-PD-1 antibody on the survival extension by SN8b is smaller (p=0.0608) and in the trend to the statistical significance. FIG. 6B depicts comparison between 12 pairs of K422-bearing mice that were treated by i.v. administration of different agents. Statistically significant difference (*, p<0.05; **, p<0.01; ***, p<0.001) in the survival is detected in the survival between individual pairs except for one pair between anti-CD79a mAb SN8b and SN8b plus anti-PD-1 antibody in which the difference is in the trend to statistical significance (NS, p=0.0608).

FIG. 7 depicts the presence of natural killer (NK) cells but absence of T and B cells in SCID mice bearing K422 human DLBCL as analyzed by flow cytometry. The control mouse is a normal BALB/c mouse. Seven other mice represent 7 groups of SCID mice bearing K422 human DLBCL that received i.v. 1) CCL130, an isotype-matched control IgG (IgG-K), 2) anti-CD79b mAb SN8, 3) SN8 and anti-PD-1 antibody, 4) anti-CD79a mAb SN8b, 5) SN8b and anti-PD-1 antibody, 6) SN8 and SN8b, and 7) SN8, SN8b and anti-PD-1 antibody, respectively. Each of the 8 groups of mice used for the flow cytometry test consisted of 4 mice but only data of a representative example of one mouse is shown for each group. Blood was collected from individual mice (4 mice/group) four times during the therapeutic experiment, i.e., pretreatment (−day 2), day 12, day 19 and day 26, but only the data of the sample collected on day 26 is shown for each group. Leucocytes in each blood sample are detected as CD45+ cells as shown in the first column. T and B cells which are detected as CD3+CD19− cells and CD3−CD19+ cells, respectively are clearly detected in the blood from the control mouse, but are absent in the blood from SCID mice of all seven groups as shown in the second column. On the other hand, NK cells that are detected as DX5+ (CD49b+; pan NK cell marker) cells and NKp46+ (activated NK cell marker) cells are present in mice of all 8 groups; it was detected throughout the blood analysis at the four time points. In the tested SCID mice, DX5+NK cells are expressed as the percentage of the CD45+CD3−CD19-cell population while NKp46+NK cells are expressed as the percentage of the CD45+CD3-CD19-DX5+ cell population. In the control normal mouse, DX5+NK cells are expressed as the percentage of the CD45+CD3−CD19-cell population while NKp46+ cells are expressed as the percentage of the CD45+CD3−CD19-DX5+ cell population. We consider that NK cells provide important antitumor effects during the immune checkpoint blockade in the SCID mice in the absence of T and B cells. In this regard, it was recently reported that NK cells express PD-1 and may contribute to immunotherapy mediated by PD-1/PD-L1 blockade (J Hsu et al., 2018, supra).

FIG. 8 depicts extension of survival of mice bearing systemic tumor of BALL-la by i. v. administration of anti-CD79b mAb SN8, anti-CD79a mAb SN8b or SN8 plus SN8b (n=7 mice per group). BALL-la is an in vivo adapted BALL-1 (Kawata et al., 1994, supra) and it expresses a high degree of CD79 (see FIG. 3B). Statistical analysis of the mouse survival was performed by Log rank test and Wilcoxon test. SN8, SN8b and SN8+SN8b are effective for the survival extension compared with control (P=0.0037, 0.0071, and 0.0004, respectively). SN8+SN8b is more effective than SN8b (P=0.0005). The difference between SN8+SN8b and SN8 is in the trend to the statistical significance (p=0.0847). The difference between SN8 and SN8b is also in the trend to the statistical significance (p=0.0778).

FIG. 9 depicts the effects of SN8 and SN8b with anti-PD-1 mAb on tumor growth (control: CCL130+anti-PD-1). The graphs show exponential growth in four groups of mice (n=8/group; see FIG. 4) for testing synergy between SN8 and SN8b with co-administration of anti-PD-1 mAb. The tumor volume data are plotted on the log scale; the tick marks correspond to the original volume in mm3. If CCL130+Anti-PD-1, SN8+Anti-PD-1, SN8b+Anti-PD-1, and SN8+SN8b+Anti-PD-1 are considered as C, A, B, and AB, then β_{A}+β_{B}-β_{C}-β_{AB}=2% per day, which is positive (p-value=5×10⁻⁵). Therefore, this result indicates that there is a statistically significant synergistic effect between the SN8 and SN8b (with co-administration of anti-PD-1 mAb).

### DETAILED DESCRIPTION

### Definitions

The term “B cell antigen receptor (BCR)” is used herein, for purposes of the specification and claims to mean a molecular complex on B cells that comprises two components, an antigen binding component and a signaling component. The BCR was initially identified on mouse B cells (Hombach et al., 1990, Nature 343:760-762). The antigen binding component is a membrane-bound form of immunoglobulin (mlg), most frequently mIgM. The signaling component comprises a disulfide-linked heterodimer, CD79a/CD79b (or called mb-1/B29 or Igα/β), that is non-covalently associated with mlg. Three mAbs, named SN8, SN8a and SN8b, define a unique covalently linked heterodimer glycoprotein complex (Okazaki et al, 1993, Blood 81:84-94). This was subsequently identified as the human CD79a (Igα; mb-1)/CD79b (IgB; B29) complex (Vasile et al., 1994, Mol. Immunol. 31:419-427; Engel et al., 1995, Leucocyte Typing V, Vol. 1, 667-670 (SF Schlossman et al, Editors) Oxford University Press, Oxford). SN8 and SN8a were identified to define individual epitopes of CD79b while SN8b appeared to define an epitope of CD79a (Okazaki et al., 1993, supra; Vasile et al., 1994, supra) that is confirmed in this disclosure. SN8 epitope was strongly expressed on non-Hodgkin's lymphoma (NHL) cells, prolymphocytic leukemia (PLL) cells and mature B acute lymphoblastic leukemia (ALL) cells compared with normal B cells (Okazaki et al., 1993, supra). Conjugates of these anti-CD79 mAbs with a cytotoxic agent effectively killed malignant B cells (Okazaki et al., 1993, supra).

The present disclosure reveals a novel molecular heterogeneity of human CD79 that is important in CD79-targeted therapy. Importance of this finding for CD79-targeted therapy is indicated by animal model studies in which CD79-expressing human B cell tumors are treated with anti-CD79b monoclonal antibody (mAb), anti-CD79a mAb or combination of the two mAbs.

The term “CD79-targeted therapy” is used herein, for purposes of the specification and claims, to mean therapy targeted to CD79 on human malignant B cells.

The term “antibody fragment” or “fragment thereof” is used herein, for purposes of the specification and claims, to mean a portion or fragment of an intact antibody molecules, wherein the fragment retains antigen-binding function; i.e., F(ab′)2, Fab′, Fab, Fv, single chain Fv (“scFv”), Fd′, Fd and any antibody fragments that bind the defined antigens. Methods for producing the various fragments from mAbs are well known to those skilled in the art (e.g., Pluckthurn, 1992, Immunol. Rev. 130:152-188; Carter, 2006, Nature Rev. Immunol. 6:343-357).

The term “immunoconjugate” is used herein, for purposes of the specification and claims, to mean a conjugate comprised of the anti-CD79b mAb or anti-CD79a mAb or a fragment thereof according to the present invention and at least one antitumor agent. Such antitumor agents are known in the art and include, but not limited to, drugs, toxins, enzymes, cytokines, radionuclides and photodynamic agents. The drugs include monomethylauristatin E (MMAE), daunorubicin and methotrexate. Toxins include ricin A chain, Pseudomonas exotoxins, diphtheria toxoin, pokeweed antiviral protein and saporin. Radionuclides include radiometals. Cytokines include interleukins, interferons, transforming growth factor (TGF)-B, tumor necrosis factors. Photodynamic agents include porphyrins and their derivatives. The methods for complexing the anti-CD79 mAbs or a fragment thereof with antitumor agents are well known to those skilled in the art (i.e., antibody conjugates as reviewed by Ghetie et al., 1994, Pharmacal. Ther. 63:209-234).

The term “chimeric antigen receptor (CAR) T cells” is used herein, for purposes of the specification and claims, to mean CAR T cells comprising antibody fragments (e.g., scFv) of anti-CD79a mAb and/or anti-CD79b mAb. The methods for producing various therapeutic CAR T cells are well known to those skilled in the art (e.g., Sadelain et al., 2017, Nature 545:423-431).

The term “isotype-matched control immunoglobulin (Ig)” is used herein, for purposes of the specification and claims, to mean a species specific (e.g. raised in the same species as the antibody to which it is compared), isotype-matched (e.g., of the same 1 g class and subclass as the antibody to which it is compared) lg which does not bind with specificity to the antigen to which the compared antibody has binding specificity, as will be more apparent from the following embodiments.

The term “mammalian host” or “host” is used herein, for purposes of the specification and claims, to mean a mouse or a human.

The term “monoclonal antibody”, as denoted as having binding specificity for an epitope of CD79a or CD79b, is used herein, for purposes of the specification and claims, to mean murine monoclonal antibodies and engineered (e.g., recombinant) antibody molecules made therefrom in which the binding specificity for an epitope of CD79a or CD79b, and includes chimeric or “humanized” antibodies, and antigen binding fragments of the described antibodies. The SN8 antibody is commercially available, such as from ThermoFisher Scientific, catalog number Catalog #604-490. The SN8 and SN8b antibodies are described in U.S. Pat. No. 5,644,033, the entire disclosure of which is incorporated herein by reference.

The term “synergy” is used herein, for purposes of the specification and claims, to mean statistically significant synergy between two drugs (or two mAbs) that was defined by Demidenko and Miller (E. Demidenko and T. W. Miller. 2019, supra). Search for synergy between two drugs has been of great interest in biological and medical research (W. R. Greco et al., 1995, Pharmacol. Reviews. 47:331-385). Previously many researchers used various in vitro assays to examine the effects of two drugs for antagonistic, additive or synergistic interaction (R. L. Momparler, 1980, Pharmacol. Therapeutics. 8:21-35). Recently Demidenko and Miller (E. Demidenko and T. W. Miller, 2019, supra) developed a method for a statistical determination of synergy between two drugs that can be applied to the in vivo assays and clinical studies in addition to the in vitro assays. In the present disclosure, we applied this method to evaluate synergy between anti-CD79a mAb SN8b and anti-CD79b mAb SN8 in the therapy of animals bearing human B cell tumors.

The term “tumor” is used herein, for purposes of the specification and claims, to mean a tumor expressing CD79a and CD79b such as non-Hodgkin's lymphoma, prolymphocytic leukemia and chronic lymphocytic leukemia.

A drawback to conventional chemotherapy and radiotherapy is the lack of selectively delivering the therapy to its intended target, diseased tissue. Monoclonal antibodies have been used to deliver therapeutics with greater target specificity, thereby reducing toxicity. Murine mAbs or fragments thereof have been used to treat human disease, often with modest to substantial clinical efficacy (see, e.g., Ghetie et al., 1994, supra). Studies show that murine monoclonal antibodies may be repeatedly given safely into patients after the antibodies are chimerized or humanized (e.g., Rosen et al., 2012, Clin. Cancer Res. 18:4820-4829; Gordon et al., 2014, Clin. Cancer Res. 20:5918-5926).

The disclosure includes the following embodiments as summarized in the following statements:

Statement 1. Synergistically enhanced tumor therapy in tumor-bearing hosts by co-administration of anti-CD79a monoclonal antibody (mAb) SN8b that is derived from hybridoma clone Q6-1D5 and anti-CD79b mAb SN8 that is derived from hybridoma clone 3A2-2E7, compared with administration of anti-CD79a mAb SN8b or anti-CD79b mAb SN8. Statement 2. Fragments of anti-CD79a mAb SN8b according to Statement 1, wherein the fragments are selected from the group consisting of F(ab′)2, Fab′, Fab, Fv, scFv, Fd′, Fd and any other antigen-binding fragments.

Statement 3. Fragments of anti-CD79b mAb SN8 according to Statement 1, wherein the fragments are selected from the group consisting of F(ab′)2, Fab′, Fab, Fv, scFv, Fd′, Fd and any other antigen-binding fragments.

Statement 4. Immunoconjugates of anti-CD79a mAb and anti-CD79b mAb according to claim 1 and antibody fragments according to Statements 2 and 3 are generated to enhance antitumor activities of antibody and antibody fragments. The immunoconjugates include conjugates of a drug, a toxin and a radionucleotide.

Statement 5. Synergistically enhanced tumor therapy in tumor-bearing hosts, with co-administration of anti-programmed cell death protein 1 (PD-1) antibody, an immune checkpoint blocker, by combined administration of anti-CD79a mAb SN8b and anti-CD79b mAb SN8, compared with administration of SN8b or SN8 in addition to anti-PD-1 antibody. Statement 6. Administration of anti-PD-1 antibody strongly potentiates antitumor activity of anti-CD79b mAb SN8, and antitumor activity of SN8+SN8b in tumor-bearing hosts.

Statement 7. Two-dimensional flow cytometry in which different leukemia-lymphoma (LL) cell lines and LL cells from different patients are simultaneously stained for CD79a and CD79b provides predictive information for efficacy of the dual targeting therapy of LL cells by simultaneously targeting of CD79a and CD79b.

Statement 8. The mAb according to Statement 1, wherein the mAb is chimeric antibody comprising a murine variable region having binding specificity for human CD79a and a human constant region of human Immunoglobulin (Ig).

Statement 9. The mAb according to Statement 1, wherein the mAb is humanized antibody comprising of murine hypervariable region having binding specificity for human CD79a, and human Ig constant region and a human Ig variable region, except for the murine hypervariable region segment.

Statement 10. Chimeric antigen receptor (CAR) T cells that contain antigen-binding fragments of anti-CD79a mAb.

Statement 11. CAR T cells that contain antigen-binding fragments of both an anti-CD79a mAb and an anti-CD79b mAb.

The following examples are presented to illustrate aspects of the disclosure are not intended to limiting in any manner.

### EXAMPLE 1

This Example provides a description of the process in which we identified the epitope defined by anti-CD79 monoclonal antibody (mAb) SN8b.

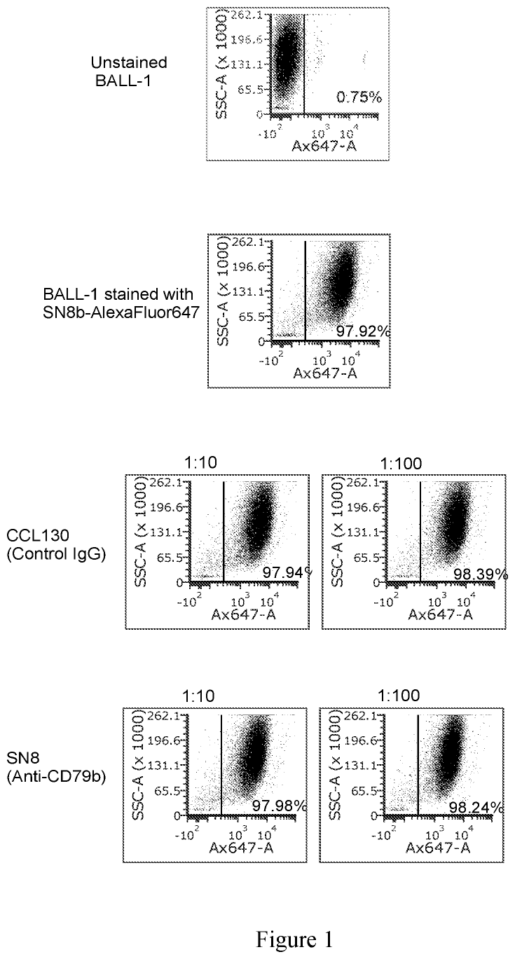

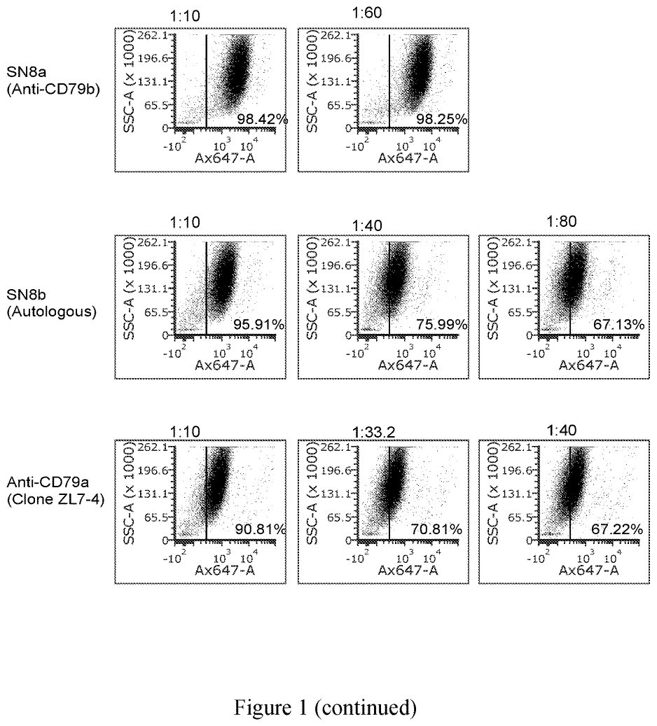

We generated three anti-CD79 mAbs, termed SN8, SN8a and SN8b (Okazaki et al., 1993, supra; Vasile et al., 1994, supra). The a-chain (or mb-1) component of the human B cell reseptor (BCR) corresponds to CD79a while the B-chain (or B29) component of the BCR corresponds to CD79b (Okazaki et al., 1993, supra; Vasile et al., 1994, supra; Engel et al, 1995, supra). SDS-PAGE analysis of the immune precipitate, Western blot analysis and competitive binding assay clearly identified that SN8 and SN8a define individual epitopes on the cell surface of CD79b. Although these analyses suggested that SN8b may defines an extracellular epitope of CD79a, additional evidence was necessary to clearly draw the conclusion. The flow cytometry analysis presented in FIG. 1 of this patent application provides such evidence.

FIG. 1 depicts a flow cytometry analysis that demonstrates specific blocking of the binding of SN8b-Alexa Fluoro 647 (SN8b-AIF1647) to CD79-expressing βALL-1 cells by incubation with serial concentrations of autologous mAb SN8b (a positive control) and anti-CD79a mAb ZL7-4 (from Invitrogen) but not by any of anti-CD79b mAb SN8, anti-CD79b mAb SN8a and an isotype-matched control IgG CCL130 (IgG1-κ). These results in addition to the previous results (Okazaki et al., 1993, supra) show that mAb SN8b defines a cell surface epitope of CD79a.

This competitive binding assay was performed by modification of our previously performed assay in which a radiolabeled mAb was used in the competitive binding assay (e.g., Matsuzaki et al, 1987, Cancer Res. 47:2160-2166). In the present assay, a radiolabeled mAb in the previous assay was replaced with a fluorochrome-labeled mAb. The current assay is briefly described below. CD79a/CD79b-positive βALL-1 cells (2×10⁶ cells/50 μl of FACS buffer) in wells of 96 well microtiter plates are incubated at 40C for 1 h with 10 μl of serial dilutions of a mAb or a control IgG. The FACS buffer consists of PBS containing 0.5% BSA and 0.5 mM EDTA. Then, 10 μl (0.25 μg) of AlexaFluor647-labeled SN8b (Ax647-SN8b) is added into individual wells and the incubation is continued for an additional 1 h at 40 C in the dark. The cells are centrifuged, washed twice with the FACS buffer, and fixed with Fix/Perm permeabilization solution (eBioscience, Thermo Fisher Scientific). The fixed cells are washed twice with the FACS buffer and transferred into BD Falcon tubes. Then the cells are analyzed by single color flow cytometry.

In the subsequent flow cytometry assays, multi-color flow cytometry was performed to test binding of AxF1647-SN8b, PE-SN8 and FITC-anti-CD45 to various human malignant hematopoietic cell lines and cells from patients. The individual cell samples were stained with Live/Dead Fixable Aqua Dead Cell Stain Kit (Invitrogen, Thermo Fisher Scientific) and results were used for gating of the graphs of the three colors flow cytometry of the individual cell lines/cell specimens. Analysis of the gated cytometry data was performed as described previously (Sribenja et al., 2021, Am. J. Cancer Res., 11:3263-3270).

### EXAMPLE 2

This Example provides a description of the initial process in which we identified the novel molecular heterogeneity of human CD79 that is the signaling component of the human BCR.

We performed sequential immunoprecipitation experiments of CD79a antigen and CD79b antigen. To this end, we prepared a cell membrane antigen preparation of B cells from a patient with B prolymphocytic leukemia (BPLL) as described previously (Okazaki et al., 1993, supra) and radiolabeled with ¹²⁵I as described previously (Okazaki et al., 1993, supra).

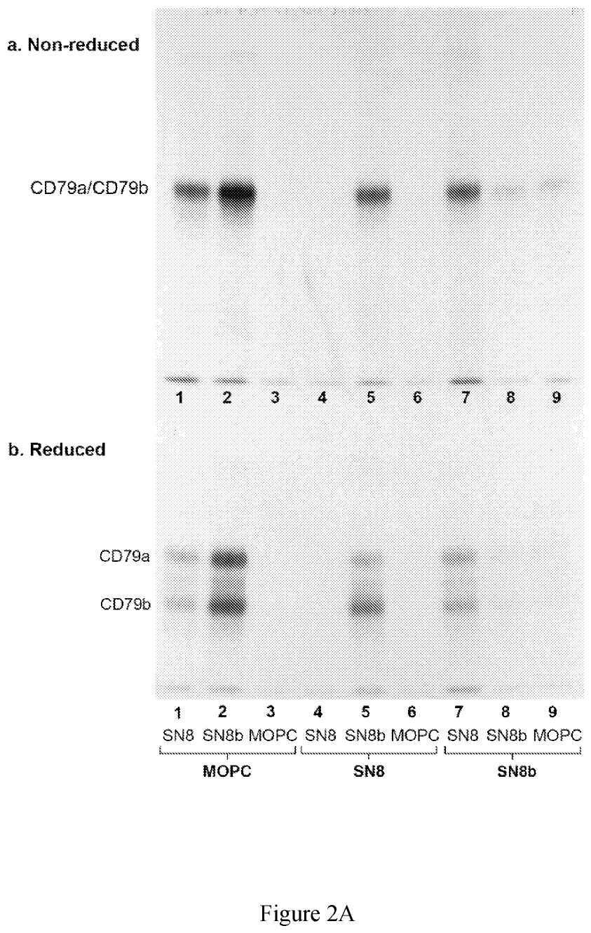

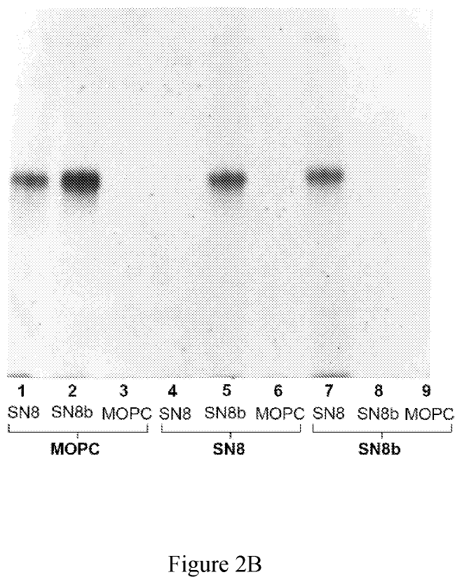

Table 1 depicts immunoprecipitation of CD79a/CD79b from ¹²⁵I-labeled BPLL antigen preparation by use of anti-CD79b mAb SN8 or anti-CD79a mAb SN8b after the ¹²⁵I-labeled antigen preparation has been pretreated with SN8-pansorbin, SN8b-pansorbin, or control IgG (MOPC)-pansorbin. Radioactivity of each immunoprecipitate is shown for the two independent experiments. The immunoprecipitated antigens are further analyzed in FIG. 2A and FIG. 2B. CD79 molecule is a covalently-linked heterodimer of CD79a molecule and CD79b molecule. Nevertheless, CD79 molecules that have been repeatedly pretreated with anti-CD79b mAb SN8-pansorbin provide a group of CD79 molecules when they are treated with anti-CD79a mAb SN8b. Similarly CD79 molecules that have been repeatedly pretreated with anti-CD79a mAb SN8b-pansorbin are still able to produce a group of CD79 molecules when they are treated with anti-CD79b mAb SN8. These results suggest the existence of molecular heterogeneity of the CD79 molecules. This conclusion is confirmed by subsequent analysis of the immunoprecipitated CD79 molecules by SDS-PAGE as shown in FIG. 2A and FIG. 2B.

TABLE 1
Immunoprecipitation of CD79a/CD79b from ¹²⁵I-labeled
BPLL antigens that were pretreated with anti-CD79b
mAb SN8, anti-CD79a mAb SN8b, or an isotype-matched
control IgG (MOPC 195v; IgG1-k).

| | | Radioactivity (cpm) of | |
|---|---|---|---|
| | | Immunoprecipitate | |
| Pretreatment | Immunoprecipitation | Experiment 1 | Experiment 2 |
| A. MOPC | 1. SN8 | 1,734 | 2,390 |
| | 2. SN8b | 5,743 | 5,786 |
| | 3. MOPC | 525 | 636 |
| B. SN8 | 1. SN8 | 411 | 468 |
| | 2. SN8b | 2,051 | 2,502 |
| | 3. MOPC | 452 | 478 |
| C. SN8b | 1. SN8 | 2,007 | 3,172 |
| | 2. SN8b | 722 | 490 |
| | 3. MOPC | 876 | 682 |

B Prolymphocytic leukemia (BPLL) cells strongly express cell surface CD79a/CD79b (mb-1/β29) (Okazaki et al., 1993, Blood 81:84-94). The BPLL antigens including CD79a/CD79b were isolated from the cell membranes of BPLL cells and labeled with ¹²⁵I as described previously (Okazaki et al., 1993, Supra), and divided into three equal aliquots. Each aliquot was subjected to pretreatment by incubating with control IgG (MOPC)-pansorbin, SN8-pansorbin, or SN8b-pansorbin. The pretreatment was repeated twice by incubating the corresponding supernate of the incubated mixture with a fresh batch of MOPC-pansorbin, SN8-pansorbin, and SN8b-pansorbin, respectively. Each of the thrice pretreated supernates was divided into three equal aliquots and subjected to immunoprecipitation with SN8-pansorbin, SN8b-pansorbin, and MOPC-pansorbin, respectively. Radioactivity of each immunoprecipitate is shown for the results of two independent experiments. The radiolabeled antigen in each immunoprecipitate were released and analyzed in SDS-PAGE as shown in FIG. 2A and FIG. 2B.

FIGS. 2A-2B depict SDS-PAGE analysis of CD79a/CD79b complex or CD79a and CD79b molecules that are generated by reduction of the disulfide bonds between CD79a and CD79b molecules. Data in Section A are those of SDS-PAGE analysis of the immunoprecipitates from Experiment 1 of Table 1 while data in Section B are those of SDS-PAGE analysis of the immunoprecipitates from Experiment 2 of Table 1. In Section A, a 85 kDa CD79a/CD79b complex (non-reduced, upper panel) or a 49 kDa CD79a molecule and a 40 kDa CD79b molecule (reduced, lower panel) are detected after immunoprecipitation with anti-CD79b mAb SN8 (panel 1) and anti-CD79a mAb SN8b (panel 2) from the control IgG (MOPC)-pansorbin pre-cleared ¹²⁵I-BPLL antigen preparation. From the SN8-pansorbin pre-cleared ¹²⁵I-BPLL antigen preparation, the CD79a/CD79b complex is not detected in the immunoprecipitate of either SN8 (lane 4) or MOPC (lane 6), whereas both CD79a/CD79b complex (non-reduced, upper panel) and CD79a and CD79b molecules (reduced, lower panel) are detected in the immunoprecipitate with SN8b (lane 5). From the SN8b-pansorbin pre-cleared ¹²⁵I-BPLL antigen preparation, the CD79a/CD79b complex is not detected in the immunoprecipitate of either SN8b (lane 8) or MOPC (lane 9), whereas both the CD79a/CD79b complex (non-reduced, upper panel) and CD79a and CD79b molecules (reduced, lower panel) are detected in the immunoprecipitate of SN8 (lane 7). These results are consistent with the results of Table 1 and show the existence of molecular heterogeneity of CD79 complex. It should be noted that anti-CD79b mAb SN8 and anti-CD79a mAb SN8b do not interfere each other in their binding with CD79 complex in the competitive binding assays (FIG. 1 of this disclosure, and; Okazaki et al., 1993, supra).

### EXAMPLE 3

This Example provides a description of the process in which we applied multi-color flow cytometry to analyze the pattern of expression of CD79a and CD79b on different human leukemia-lymphoma (LL) cell lines and LL cells from different patients. This multi-color assay provides a method to estimate the probability of success in the CD79a/CD79b dual targeting therapy of human leukemia-lymphoma (HLL) patients.

Various established human hematopoietic cell lines were cultured in RPMI 1640 medium supplemented with 4 to 10% fetal bovine serum (FBS), penicillin (100 U/ml) and streptomycin (50 μg/ml) as described previously (Okazaki et al., 1993, supra; Kawata et al., 1994, supra). The following 8 cell lines were originally provided to us by Jun Minowada of our Institutional cell culture laboratory and have been extensively used by us and others (e.g., Okazaki et al., 1993, supra; Kawata et al., 1994, supra). These are SU-DHL-4 (germinal center B-cell-like diffuse large B-cell lymphoma, GCB-DLBCL), Daudi (Burkitt's lymphoma, BL), Ramos (BL), βALM-3 (lymphoma), U698-M (lymphoma), βALL-1 (mature B acute lymphoblastic leukemia, B ALL), KM-3 (immature B-lineage ALL), MOLT-4 (T ALL). They were authenticated by gene mapping at the Genomic Shared Resources of our Institute. Among the additional cell lines, MO1043 (chronic lymphocytic leukemia, CLL) was established in our laboratory in collaboration with Tin Han (Kawata et al., 1993, supra). βALL-1a was established in our laboratory by in vivo adaptation of βALL-1 (Kawata et al., 1994, supra). Karpas-422 (K422; GCB-DLBCL) and TMD8 (activated B-cell-like DLBCL, ABC-DLBC) were obtained from Leibniz Institute/DSMZ German Collection of Microorganisms and Cell Cultures through C Mavis and F J Hernandez-Ilizaliturri.

Peripheral blood, spleens and lymph nodes from cancer patients were obtained. Mononuclear cells and blast cells were isolated from the cell suspensions of these specimens by centrifugation on a Ficoll-Paque gradient.

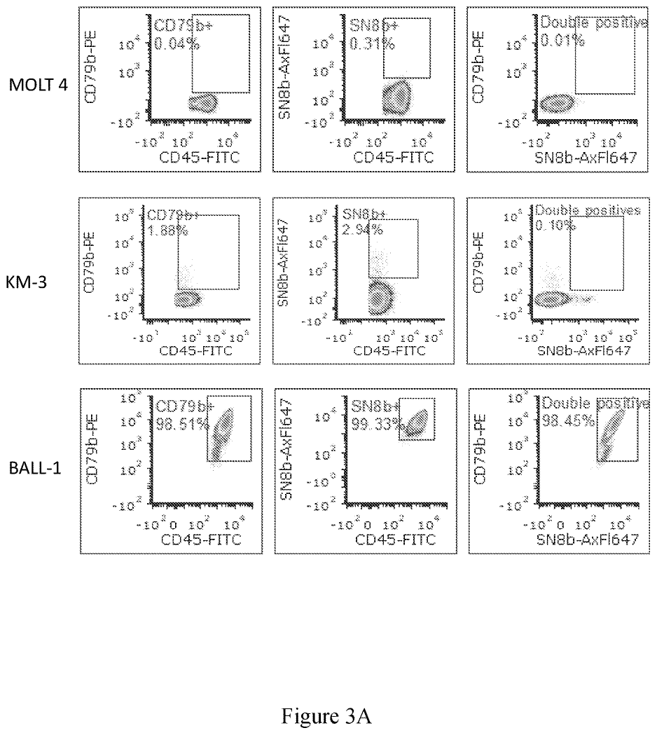

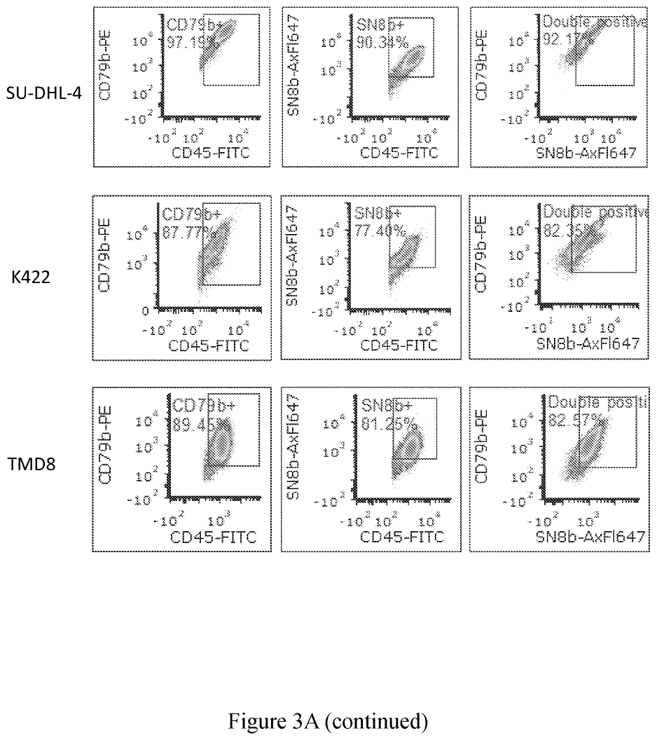

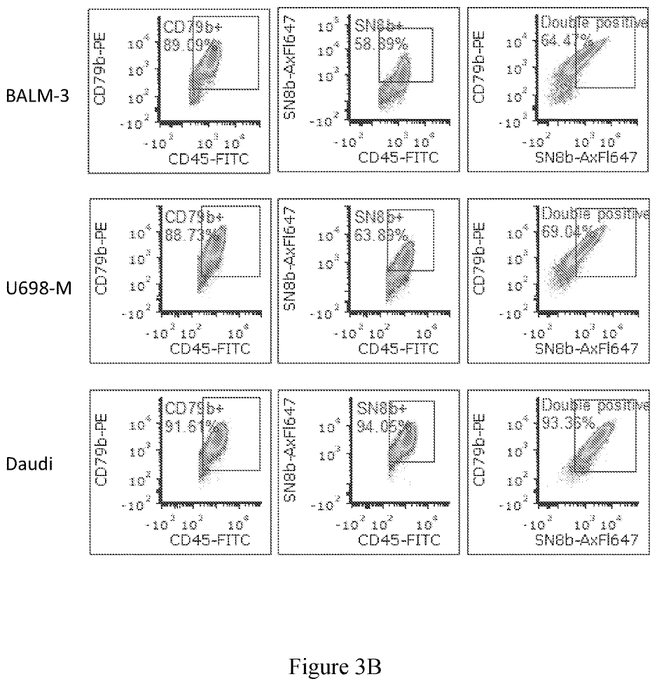

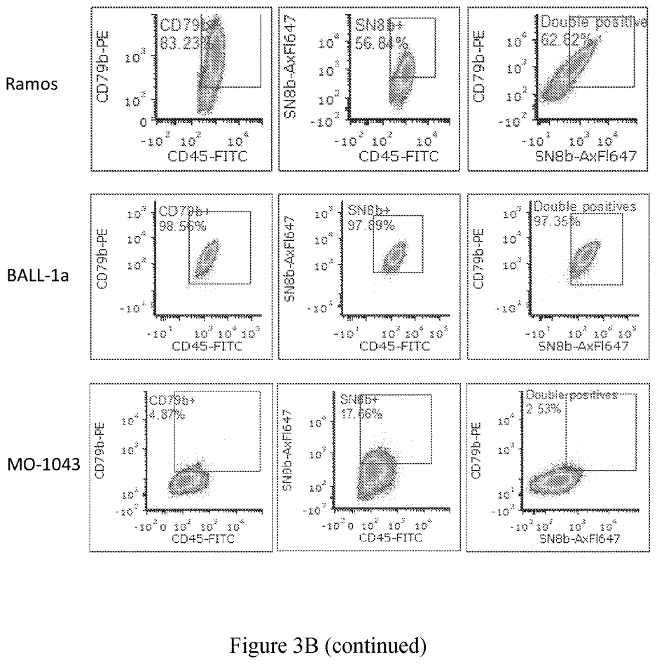

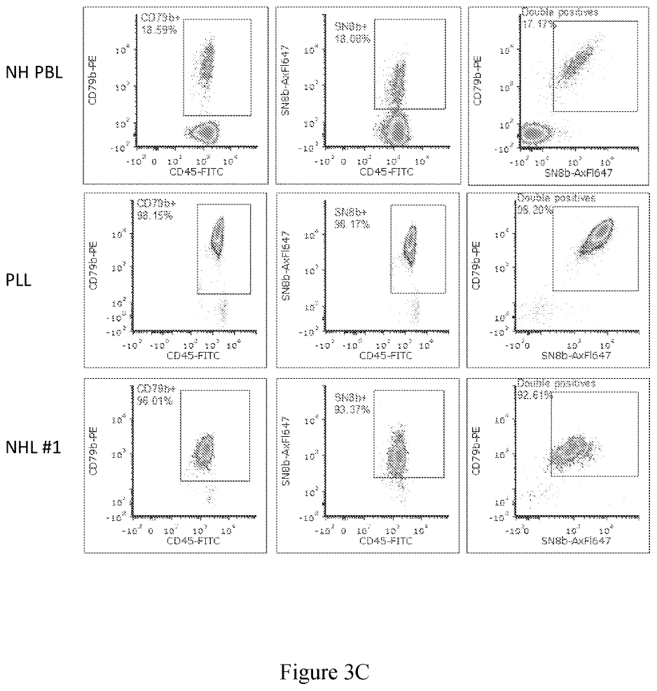

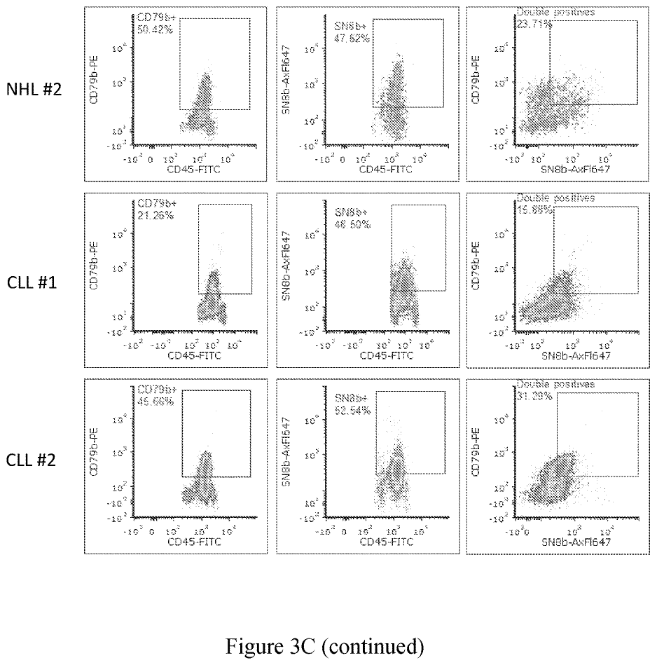

FIG. 3A depicts expression of CD79a and CD79b on 6 LL cell lines that include 1) MOLT-4 T-lineage acute lymphoblastic leukemia (T ALL) cell line, 2) KM-3 immature B-lineage ALL (B ALL) cell line, 3) βALL-1 mature B ALL cell line, 4) SU-DHL-4 diffuse large B cell lymphoma (DLBCL) cell line, 5) Karpas-422 (K422) DLBCL cell line and 6) TMD8 DLBCL cell line. Rreactivity of anti-CD79b mAb SN8 and anti-CD79a mAb SN8b with these LL cell lines are analyzed in a two-dimensional flow cytometry. For the present analysis, mAb SN8 was labeled with phycoerythrin (PE) while mAb SN8b was labeled with Alexa Fluoro 647 (AxF1647). FITC-labeled anti-CD45 mAb (from eBioscience) is included in the assay as a leucocyte marker. Neither SN8 nor SN8b shows significant reactivity with MOLT-4 T cells and their reactivities with KM-3 cells are minor (<3%). In contrast, both SN8 and SN8b reacted with the majority of cells of βALL-1, SU-DHL-4, K422 and TMD8 (i.e., >90% for βALL-1 and SU-DHL-4, and >77% for K422 and TMD8). In a simultaneous staining experiment with SN8 and SN8b, the majority of these four cell lines reacted with both SN8 and SN8b. However, these results do not indicate that anti-CD79b mAb SN8 and anti-CD79a mAb SN8b react with CD79b and CD79a of the same CD79 molecules, but the results of Table 1 and FIGS. 2A-2B indicate that SN8 and SN8a are reacting with CD79b and CD79a of different CD79 molecules. It should be noted that βALL-1 cells express 5.9×10+SN8 epitopes and 5.7×10⁴ SN8b epitopes per cell on the cell surface (Okazaki et al., 1993, supra).

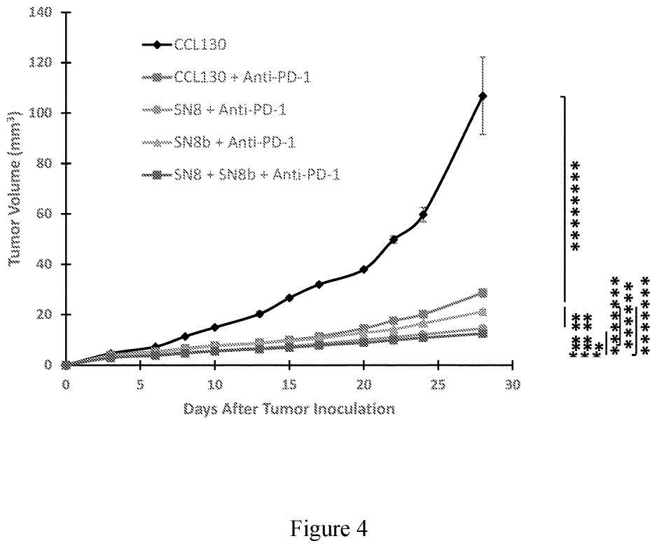

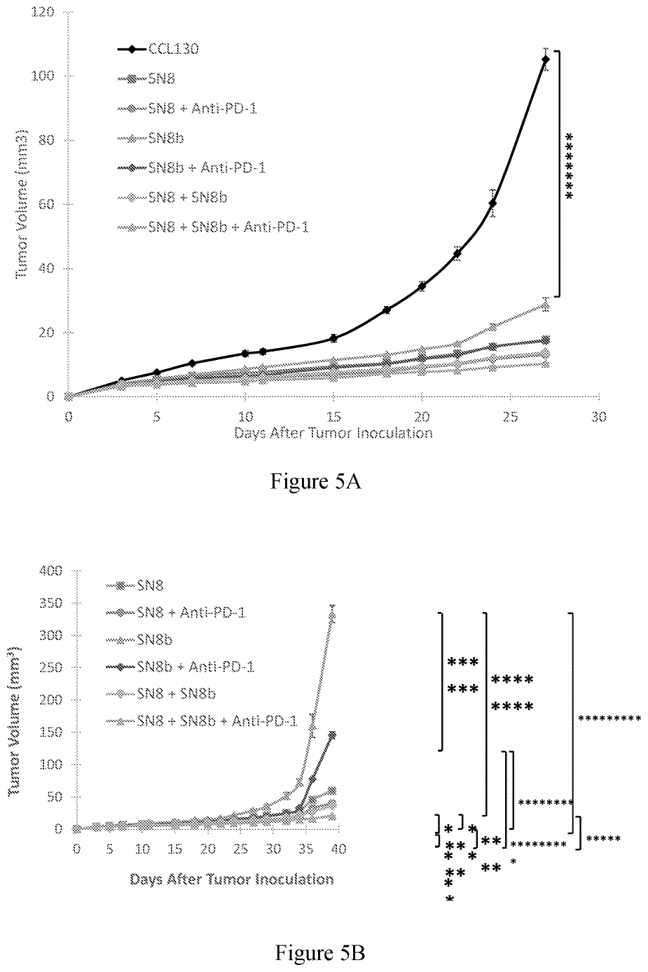

FIG. 3B depicts the continued tests of reactivity of PE-labeled anti-CD79b mAb SN8 and AxF1647-labeled anti-CD79a mAb SN8b with 6 additional human leukemia-lymphoma cell lines; in the assay FITC-labeled anti-CD45 mAb is included as a positive control for the leucocytes. These 6 cell lines include βALM-3 (B lymphoma), U698-M (B lymphoma), Daudi (Burkitt's lymphoma), Ramos (Burkitt's lymphoma), βALL-la (in vivo adapted βALL-1) and MO-1043 (B chronic lymphocytic leukemia, B CLL). The percentage of the SN8b-positive cells is lower than that of the SN8-positive cells for βALM-3, U698-M and Ramos. Consequently, the percentage of the CD79b/CD79a double positive cells for these 3 cell lines is 64.47%, 69.04% and 62.82%, respectively which is similar to the levels of SN8b-positive cells, but is lower than that of SN8-positive cells, i.e., 89.09%, 88.73% and 83.23%, respectively. Dual targeting of CD79a and CD79b in these malignant B cells will be less effective compared with the dual targeting of βALL-1, SU-DHL-4, K422 AND TMD8 of FIGS. 3A-3B, and also compared with the dual targeting of Daudi and βALL-la of FIG. 4. In contrast, Daudi expresses a similar level of CD79b (91.61%) and CD79a (94.05%) and consequently expresses a similar level (93.36%) of double-positive cells. βALL-la, an in vivo adapted βALL-1 (Kawata et al., 1994, Cancer Res. 54:2688-2694), express similar levels of CD79b and CD79a to βALL-1 (see FIGS. 3A-3B). MO1043 is an established B CLL cell line (Kawata et al., 1993, Leukemia Res. 17:883-894) and express low levels of CD79b and CD79a.

FIG. 3C depicts reactivity of PE-labeled anti-CD79b mAb SN8, Alexa Fluoro 647 (AxF1647)-labeled anti-CD79a mAb SN8b and FITC-labeled anti-CD45 mAb (control) with normal human peripheral blood lymphocytes (NH PBL), prolymphocytic leukemia (PLL) lymphocytes, non-Hodgkin's lymphoma (NHL) lymphocytes from 2 patients (#1 and 2) and chronic lymphocytic leukemia (CLL) lymphocytes from 2 patients (#1 and 2). The PE-SN8 and the AxF1647-SN8b react with 98.15% and 98.17% of the PLL cells, respectively, and the same number (98.20%) of the cells are double positive for SN8 (anti-CD79b) and SN8b (anti-CD79a). Over 90% of the No. 1 NHL cells reacts with PE-SN8 and AxF1647-SN8b, respectively, and the similar number (92.61%) of the cells are double positive for SN8 and SN8b. In contrast, only 50.42% and 47.62% of the No. 2 NHL cells react with PE-SN8 and AxF1647-SN8b, respectively, and only 23.71% of the NHL cells are double positive for SN8 and SN8b. The results indicate that #1 NHL cells can be targeted better by dual targeting with SN8 and SN8b compared with #2 NHL cells. These results indicate that more than a half of the CD79-positive No. 2 NHL cells express only SN8b-defined epitope of CD79a or only SN8-defined epitope of CD79b. Anti-CD79a AxF1647-SN8b reacts with higher percentage of CLL cells (#1 and 2) than anti-CD79b PE-SN8 (48.50% vs 21.26% for #1 and 52.54% vs 45.66% for #2) and the number of the double-positive cells is lower than that of the SN8b-positive cells, i.e., 15.88% for #1 and 31.29% for #2.

### EXAMPLE 4

This Example demonstrates that anti-CD79a mAb SN8b is capable of suppression of growth of B cell tumors in mice and extend survival of the tumor-bearing mice. Furthermore, the combination of anti-CD79b mAb SN8 and anti-CD79a mAb SN8b shows synergistic enhancement of antitumor activity compared with therapy with SN8 or SN8b alone. Furthermore, combination of anti-CD79 mAb(s) with anti-programmed cell death protein 1 (PD-1) antibody, an immune checkpoint inhibitor, strongly potentiates antitumor activity compared with therapy with anti-CD79 mAb(s) or anti-PD-1 antibody.

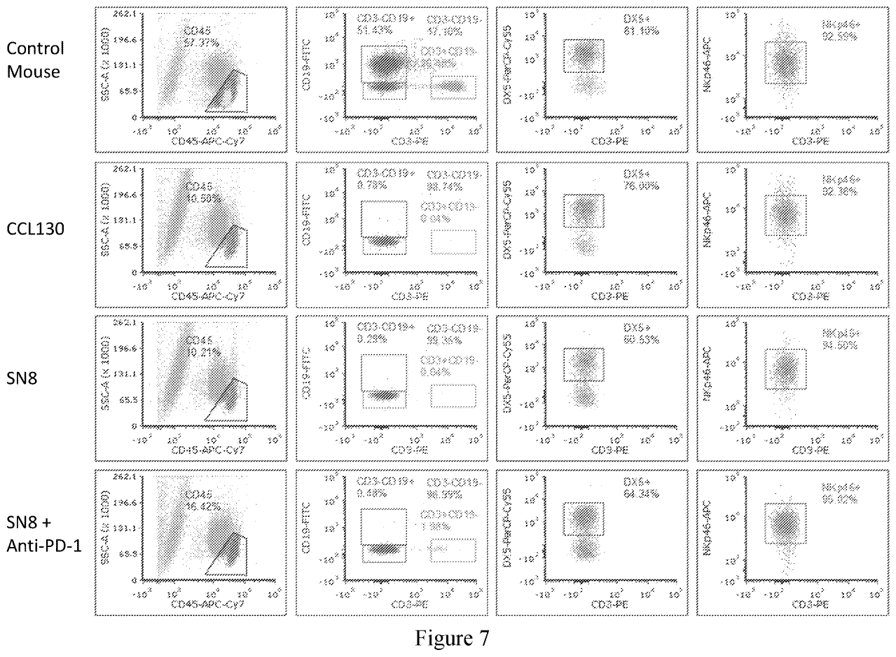

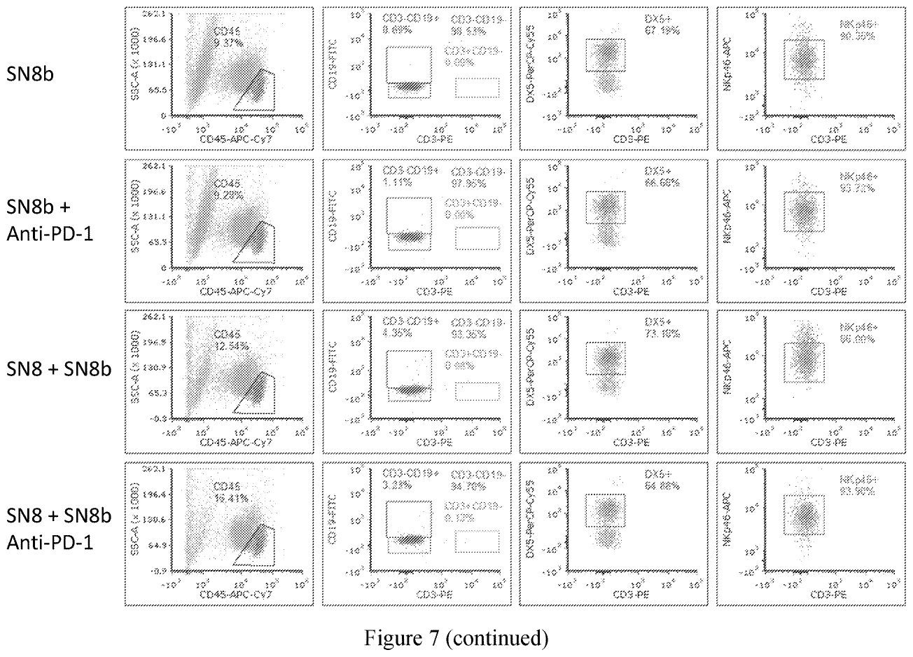

FIG. 4 depicts synergistic suppression of growth of K422 human DLBCL tumor by combined use of anti-CD79 mAbs and anti-programmed cell death protein 1 (PD-1) antibody, an immune checkpoint inhibitor. The antitumor activities of anti-CD79 mAbs and anti-PD-1 antibody are measured by decrease in tumor size after intravenous (i.v.) administration of mAbs and antibody into tumor-bearing SCID mice. Immune checkpoint blockade strategies for tumor therapy including anti-PD-1 antibody therapy are known to involve modulation of cytotoxic T cell activation against tumor cells (Topalian et al., 2012, supura; Pardoll, 2012; supra; Ribas, 2012, supra; Wei et al., 2017, supra; Moral et al, 2021, supra). On the other hand, SCID mice are deficient of T and B cells (Bosma et al., 1983, supra). We (see FIG. 7) and others (Welsh et al., 1991, J. Exp. Med., 173:1053-1063) found that SCID mice possess natural killer (NK) cells which may contribute to PD-1/PD-L1 blockade (Hsu et al., 2018, supra). In the present and the following studies, we found that anti-PD-1 antibody shows strong antitumor activity in SCID mice bearing human tumors. Analysis of synergy by dual targeting of CD79a and CD79b of B cell tumor was performed with the help of the Bioanalytics, Metabolomics and Pharmacokinetics (BMPK) Shared Resource of Roswell Park Comprehensive Cancer Center. The synergy analysis was performed by use of the statistical determination of synergy (Demidenko and Miller, 2019, PLOS ONE 14 (11): e0224137). The analysis demonstrated that anti-CD79a mAb SN8b+anti-CD79b mAb SN8 show statistically significant synergistic antitumor activity compared with SN8b alone and SN8 alone in the presence of anti-PD-1 antibody (P=0.00005). Furthermore, anti-PD-1 antibody strongly potentiates antitumor activities of SN8b, SN8 and SN8b+SN8, respectively.

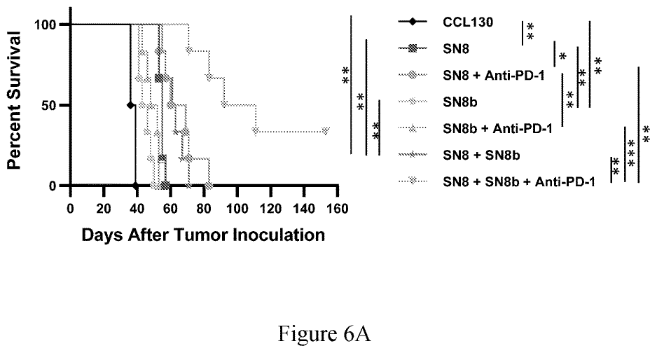

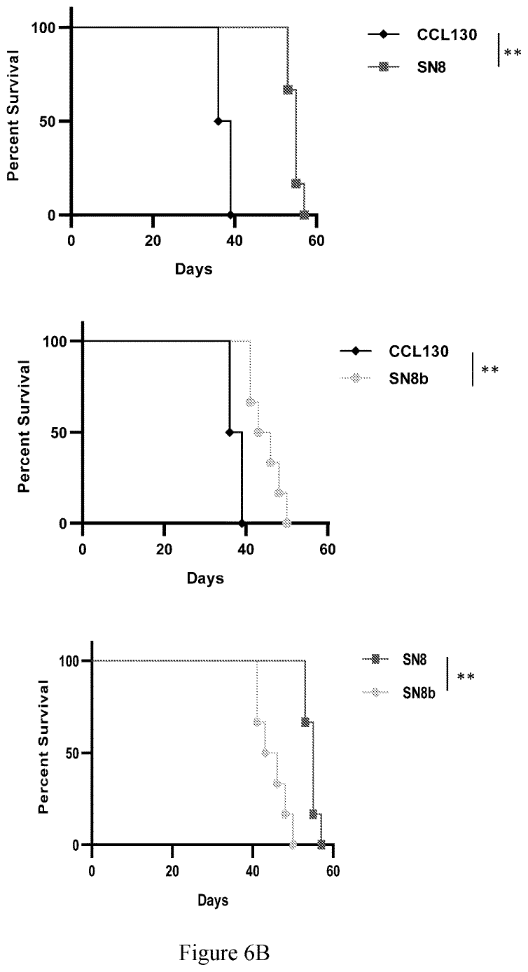

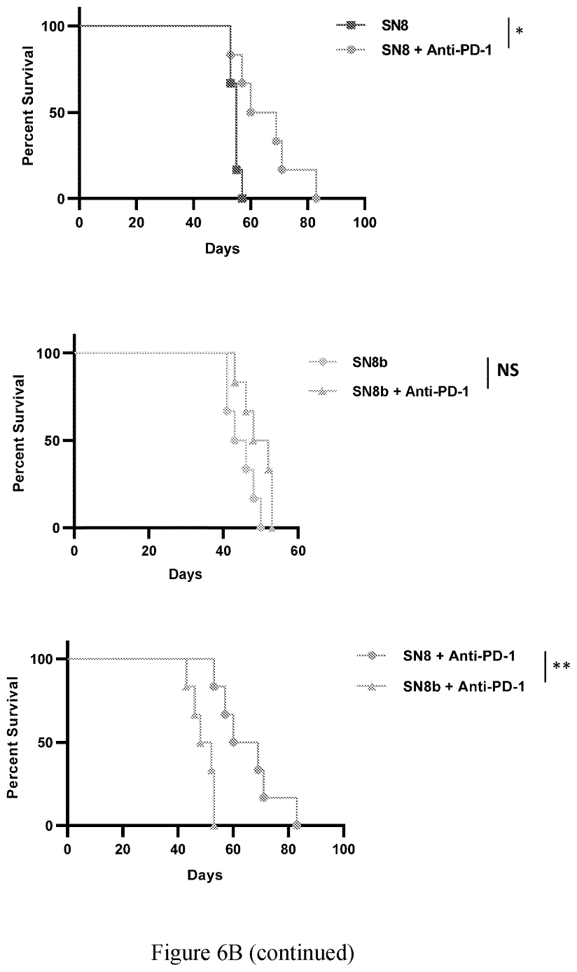

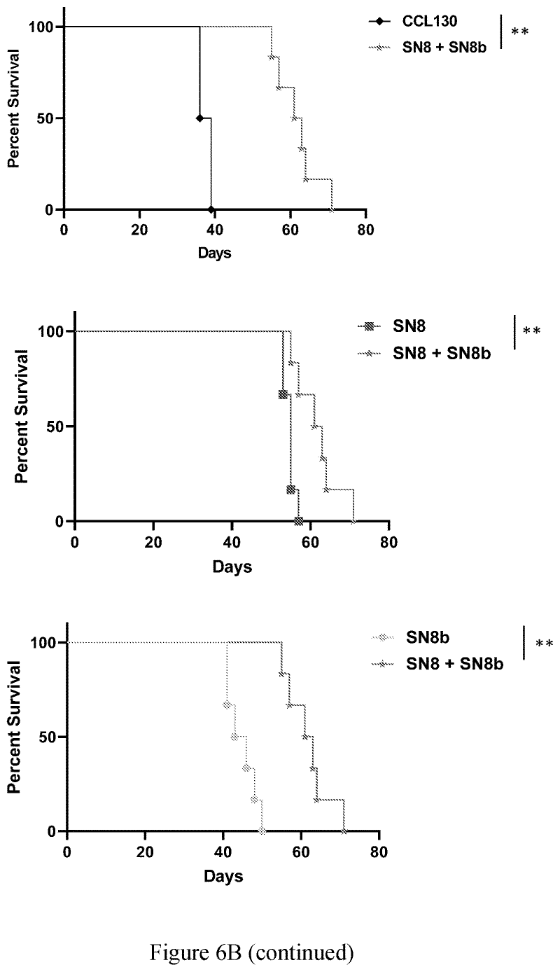

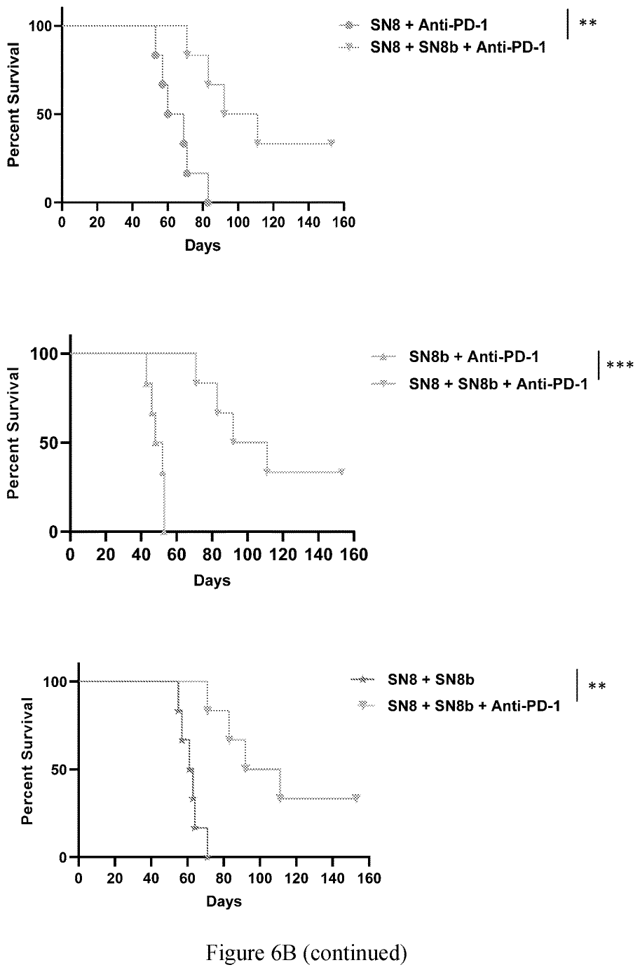

FIGS. 6A-6B depict synergistic effect by combined administration of anti-CD79a mAb SN8b and anti-CD79b mAb SN8 into SCID mice bearing K422 human DLBCL on the extension of survival.

FIG. 6A depicts extension of survival of K422 tumor bearing mice by intravenous (i.v.) administration of SN8b, SN8 or SN8b+SN8 in the absence or in the presence of anti-PD-1 antibody, an immune checkpoint blocker. CCL130 is an isotype-matched control IgG (IgG1-κ). Combined administration of SN8b and SN8 strongly enhanced the extension of survival compared with administration of SN8b alone or SN8 alone in the absence of anti-PD-1 antibody (P<0.01, ** and p<0.01, **, respectively). Furthermore, SN8b and SN8 acted synergistically in the extension of the survival as demonstrated by statistical determination of synergy (Demidenko and Miller, 2019, supra) (P=0.03; Control: CCL130). Addition of anti-PD-1 antibody to SN8 or SN8+SN8b enhanced the extension of survival (* P<0.05 and*P<0.01, respectively). The effect of anti-PD-1 antibody on the survival extension by SN8b is smaller (p=0.0608) and in the trend to the statistical significance (see below, FIG. 6B).

FIG. 6B depicts comparison between 12 pairs of K422-bearing mice that were treated by i.v. administration of different agents. Statistically significant difference (*, p<0.05; **, p<0.01; ***, p<0.001) in the survival is detected in the survival between individual pairs except for one pair between anti-CD79a mAb SN8b and SN8b plus anti-PD-1 antibody in which the difference is in the trend to statistical significance (NS, p=0.0608).

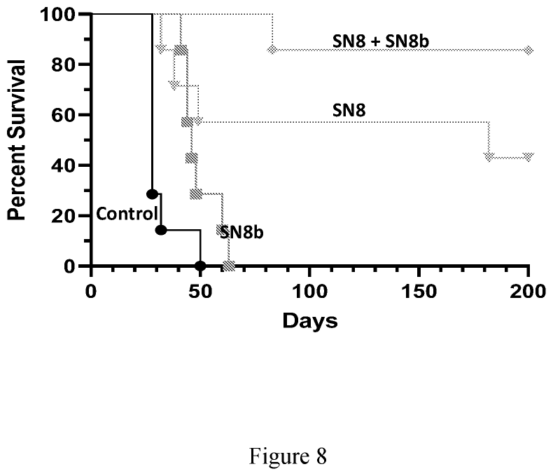

FIG. 8 depicts extension of survival of mice bearing systemic tumor of βALL-la by i. v. administration of anti-CD79b mAb SN8, anti-CD79a mAb SN8b or SN8 plus SN8b. βALL-la is an in vivo adapted βALL-1 (Kawata et al., 1994, supra) and it expresses a high degree of CD79 (see FIG. 3B).

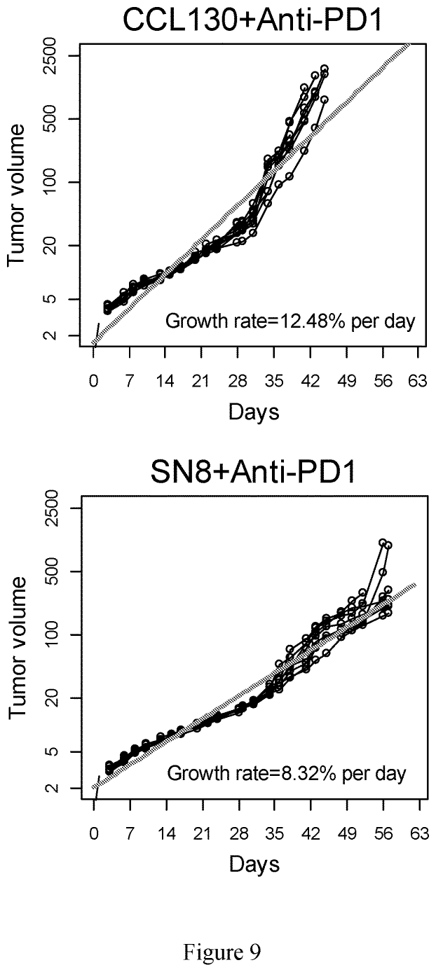

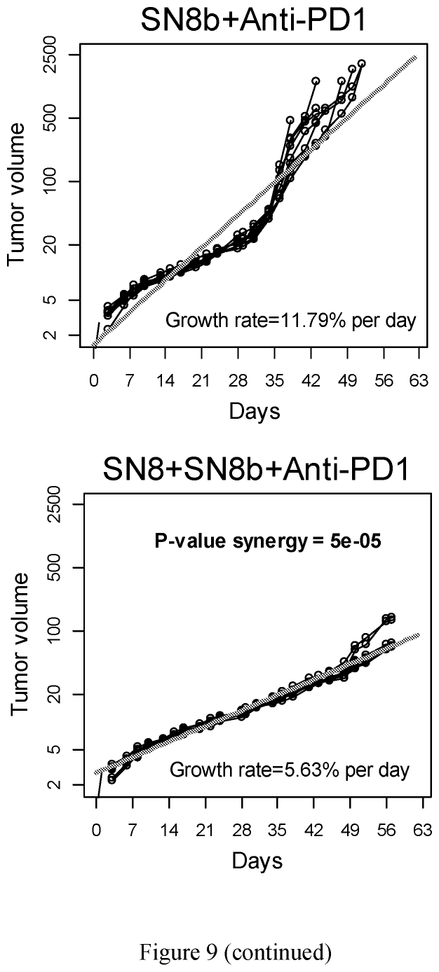

FIG. 9 shows a synergistic inhibition of tumor growth using a combination of SN8, SN8b, and an Anti-PD-1 antibody.

1. A method comprising co-administration of anti-CD79a monoclonal antibody (mAb) SN8b, anti-CD79b mAb SN8, or antigen binding fragments thereof, to thereby produce synergistically enhanced tumor therapy in tumor-bearing hosts relative to administration of anti-CD79a mAb SN8b or anti-CD79b mAb SN8 alone.

2. The method of claim 1, wherein the fragments of anti-CD79a mAb SN8b are selected from the group consisting of F(ab′)₂, Fab′, Fab, Fv, scFv, Fd′, Fd and any other antigen-binding fragments.

3. The method of claim 1, wherein the fragments of anti-CD79b mAb SN8 are selected from the group consisting of F(ab′)2, Fab′, Fab, Fv, scFv, Fd′, Fd and any other antigen-binding fragments.

4. The method of claim 1, wherein the anti-CD79a mAb and/or anti-CD79b or fragments thereof comprise immunoconjugates of that enhance antitumor of said antibodies or fragments thereof, and wherein said immunoconjugates comprise the anti-CD79a mAb and/or anti-CD79b or fragments thereof and a drug, a toxin, or a radionucleotide.

5. A method comprising co-administration to an individual in need thereof of an anti-programmed cell death protein 1 (PD-1) antibody, and anti-CD79a monoclonal antibody (mAb) SN8b and mAb SN8, to thereby synergistically enhance tumor therapy in an individual who has a tumor, relative to administration of either SN8b or SN8 alone with the anti-PD-1 antibody.

6. A method comprising analyzing a sample from a patient to determine the presence of CD79a and CD79b positive cancer cells and administering to the patient a combination of an anti-CD79a monoclonal antibody (mAb) and an anti-CD79b mAb or antigen binding fragments thereof, the method optionally further comprising administering to the individual an anti-PD-1 antibody.

7. The method of claim 6, wherein the cancer cells comprise B cell cancer cells.

8. Chimeric antigen receptor (CAR) T cells that contain antigen-binding fragments of an anti-CD79a mAb.

9. Chimeric antigen receptor (CAR) T cells that contain antigen-binding fragments of both an anti-CD79a mAb and an anti-CD79b mAb.
